(19) United States
(12) Patent Application Publication
(10) Pub. No.: US 2026/0311845 A1
(43) Pub. Date: Oct. 8, 2026

(54) MONITORING AND IMPROVING MENTAL HEALTH OUTCOMES VIA CONVERSATIONAL ARTIFICIAL INTELLIGENCE

(71) Applicant: Texas State University, San Marcos, TX (US)

(72) Inventors: Anandi Dutta, San Marcos, TX (US); Shivani Mruthyunjaya, Georgetown, TX (US)

(21) Appl. No.: 19/637,194

(22) Filed: Apr. 2, 2026

Related U.S. Application Data
(60) Provisional application No. 63/782,891, filed on Apr. 3, 2025.

(51) Int. Cl. G16H 20/70

(52) U.S. Cl. CPC G16H 20/70

(57) ABSTRACT

A computer-implemented method, system, and computer program product for providing augmented mental health support. A conversational input is received from a user via a conversational interface module. The conversational input is analyzed to identify a sentiment and/or a mental health indicator, which corresponds to a specific clinical metric or behavioral marker, such as a measured level of stress, anxiety, or depression, identified within a conversational input to characterize a user's current psychological state. Longitudinal historical data associated with the user is then retrieved from a database, where the longitudinal historical data includes data points from the previous conversational sessions. A personalized response is then generated based on the identified mental health indicator and the retrieved longitudinal historical data. The personalized response is then transmitted to a user interface. The longitudinal historical data in the database is then updated to include the mental health indicator identified in the current session.

### TECHNICAL FIELD

The present disclosure relates generally to digital mental health solutions, and more particularly to a conversational artificial intelligence (AI) system for providing mental health support through personalized engagement, longitudinal progress tracking, and expert-integrated data analytics.

### BACKGROUND

Digital mental health solutions, and specifically conversational artificial intelligence (AI) agents, have emerged as a promising means to address significant barriers in mental health care. Traditional therapy often faces challenges, such as high costs, long wait times for appointments, limited availability of practitioners, and persistent social stigma that may prevent individuals from seeking help. Conversational AI, typically implemented as chatbots, has the potential to bridge the gap between mental health needs and available resources by providing 24/7 accessible, affordable, and scalable assistance.

Despite these advantages, existing mental health AI models often suffer from critical limitations that hinder their therapeutic effectiveness. Many current systems utilize a “one-size-fits-all” approach that lacks personalized engagement and fails to track a user's progress over time. Furthermore, typical conversational agents often lack genuine empathy and emotional nuance, which can lead to slow or unnatural responses that alienate users, especially during acute crises or complex mental health emergencies.

Moreover, existing technologies often operate as isolated tools rather than integrated components of a broader clinical workflow. There is frequently a lack of “human-in-the-loop” oversight, meaning that valuable insights from chat histories are not effectively shared with professional therapists to augment their treatment plans. Concerns regarding data privacy, potential algorithmic bias, and a lack of longitudinal studies further complicate the trustworthiness and safety of these digital interventions.

Consequently, there is a need for a comprehensive conversational AI framework that provides human-like, personalized support while maintaining a high standard of clinical alignment and ethical safety. Such a system would ideally integrate expert therapist feedback, offer science-backed coping strategies, and utilize data-driven insights to help both users and practitioners track mental health journeys over time without replacing the essential role of the human therapist.

### SUMMARY

In one embodiment of the present disclosure, a computer-implemented method for providing augmented mental health support comprises receiving a conversational input from a user during a current session. The method further comprises analyzing the conversational input using a large language model (LLM) to identify a mental health indicator. The method additionally comprises retrieving, from a database, longitudinal historical data associated with the user, where the longitudinal historical data comprises data points from a plurality of previous conversational sessions. Furthermore, the method comprises generating a personalized response based on a combination of the identified mental health indicator and the retrieved longitudinal historical data. Additionally, the method comprises transmitting the personalized response to a user interface. In addition, the method comprises updating the longitudinal historical data in the database to include the mental health indicator identified in the current session.

Other forms of the embodiment of the computer-implemented method described above are in a system and in a computer program product.

The foregoing has outlined rather generally the features and technical advantages of one or more embodiments of the present disclosure in order that the detailed description of the present disclosure that follows may be better understood. Additional features and advantages of the present disclosure will be described hereinafter which may form the subject of the claims of the present disclosure.

### BRIEF DESCRIPTION OF THE DRAWINGS

A better understanding of the present disclosure can be obtained when the following detailed description is considered in conjunction with the following drawings, in which:

FIG. 1 illustrates an embodiment of the present disclosure of a computing environment for practicing the principles of the present disclosure;

FIG. 2 is a diagram of the software components used by the computer to monitor and improve mental health outcomes via conversational artificial intelligence in accordance with an embodiment of the present disclosure;

FIG. 3 is a flowchart of a method for monitoring and improving mental health outcomes via conversational artificial intelligence in accordance with an embodiment of the present disclosure; and

FIG. 4 is a flowchart of a method for implementing an exemplary user interface (UI) view of a longitudinal mental health dashboard in accordance with an embodiment of the present disclosure.

### DETAILED DESCRIPTION

As stated above, digital mental health solutions, and specifically conversational artificial intelligence (AI) agents, have emerged as a promising means to address significant barriers in mental health care. Traditional therapy often faces challenges, such as high costs, long wait times for appointments, limited availability of practitioners, and persistent social stigma that may prevent individuals from seeking help. Conversational AI, typically implemented as chatbots, has the potential to bridge the gap between mental health needs and available resources by providing 24/7 accessible, affordable, and scalable assistance.

Despite these advantages, existing mental health AI models often suffer from critical limitations that hinder their therapeutic effectiveness. Many current systems utilize a “one-size-fits-all” approach that lacks personalized engagement and fails to track a user's progress over time. Furthermore, typical conversational agents often lack genuine empathy and emotional nuance, which can lead to slow or unnatural responses that alienate users, especially during acute crises or complex mental health emergencies.

Moreover, existing technologies often operate as isolated tools rather than integrated components of a broader clinical workflow. There is frequently a lack of “human-in-the-loop” oversight, meaning that valuable insights from chat histories are not effectively shared with professional therapists to augment their treatment plans. Concerns regarding data privacy, potential algorithmic bias, and a lack of longitudinal studies further complicate the trustworthiness and safety of these digital interventions.

Consequently, there is a need for a comprehensive conversational AI framework that provides human-like, personalized support while maintaining a high standard of clinical alignment and ethical safety. Such a system would ideally integrate expert therapist feedback, offer science-backed coping strategies, and utilize data-driven insights to help both users and practitioners track mental health journeys over time without replacing the essential role of the human therapist.

The embodiments of the present disclosure provide a means for augmenting mental health support through a clinically-integrated, conversational artificial intelligence (AI) framework that bridges the gap between digital intervention and professional therapy. By utilizing a fine-tuned, instruction-based large language model (LLM), the embodiments of the present disclosure provide empathetic, personalized responses to user inputs while simultaneously identifying mental health indicators and sentiment. Unlike conventional “one-size-fits-all” chatbots, the embodiments of the present disclosure maintain a longitudinal database of user progress, allowing for the identification of cross-session patterns and the generation of data-driven insights, such as calendar heatmaps and symptom intensity plots. These insights are specifically designed to be shared with professional therapists via an expert interface, thereby enhancing the therapeutic workflow without replacing the human practitioner.

Furthermore, embodiments of the present disclosure provide a robust safety and ethical framework through the integration of a dedicated crisis detection module and an anonymization engine. The crisis detection module continuously monitors conversational inputs for high-risk indicators to trigger immediate intervention protocols, such as providing emergency resources or direct practitioner notification. A high-risk indicator (also referred to herein as the “high-risk mental health indicator”), as used herein, refers to a specific sentiment or mental health metric identified within a user's conversational input that signifies an acute crisis or emergency requiring the immediate execution of a crisis intervention protocol. A crisis intervention protocol, as used herein, is a programmed sequence of safety actions, such as providing emergency contact resources or sending direct notifications to a human-in-the-loop professional, that is automatically executed by the system upon the detection of a high-risk mental health indicator within a user's conversational input. Furthermore, embodiments of the present disclosure incorporate a “human-in-the-loop” feedback mechanism that allows professional therapists to evaluate AI-generated responses. This evaluative data is used to further tune the LLM, ensuring that the conversational agent remains aligned with evolving clinical standards and therapeutic best practices. By combining long-term progress tracking with expert oversight and advanced natural language processing (NLP) techniques, embodiments of the present disclosure provide a secure, scalable, and clinically-sound solution for modern mental health management. A further discussion regarding these and other features is provided below.

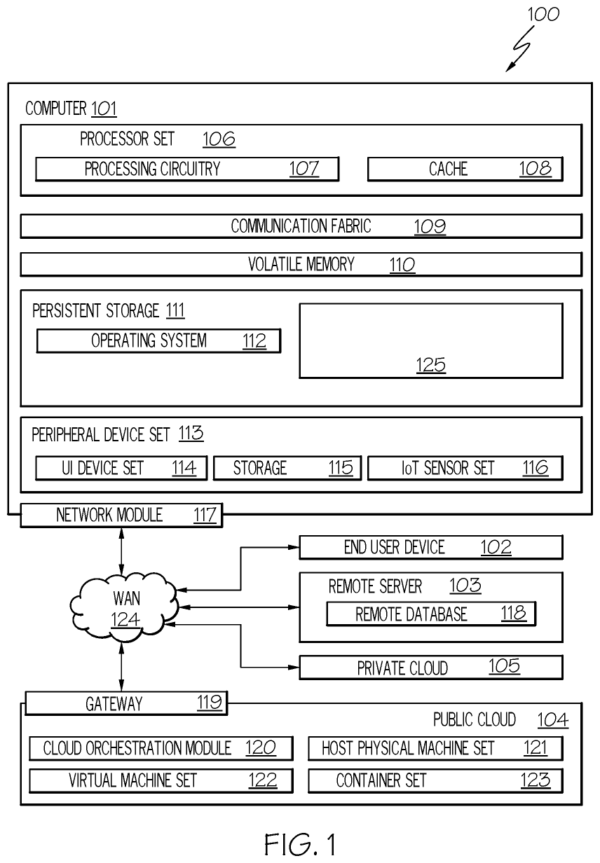

Referring now to the Figures in detail, FIG. 1 illustrates an embodiment of the present disclosure of a computing environment 100 for practicing the principles of the present disclosure.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code (stored in block 125) involved in performing the inventive methods, such as monitoring and improving mental health outcomes via conversational artificial intelligence. In addition to block 125, computing environment 100 includes, for example, computer 101 (also referred to herein as computing device 101), network 124, such as a wide area network (WAN), end user device (EUD) 102, remote server 103, public cloud 104, and private cloud 105. In this embodiment, computer 101 includes processor set 106 (including processing circuitry 107 and cache 108), communication fabric 109, volatile memory 110, persistent storage 111 (including operating system 112 and block 125, as identified above), peripheral device set 113 (including user interface (UI) device set 114, storage 115, and Internet of Things (IoT) sensor set 116), and network module 117. Remote server 103 includes remote database 118. Public cloud 104 includes gateway 119, cloud orchestration module 120, host physical machine set 121, virtual machine set 122, and container set 123.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 118. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 106 includes one, or more, computer processors of any type now known or to be developed in the future. Processing circuitry 107 may be distributed over multiple packages, for example, multiple, coordinated integrated circuit chips. Processing circuitry 107 may implement multiple processor threads and/or multiple processor cores. Cache 108 is memory that is located in the processor chip package(s) and is typically used for data or code that should be available for rapid access by the threads or cores running on processor set 106. Cache memories are typically organized into multiple levels depending upon relative proximity to the processing circuitry. Alternatively, some, or all, of the cache for the processor set may be located “off chip.” In some computing environments, processor set 106 may be designed for working with qubits and performing quantum computing.

Computer readable program instructions are typically loaded onto computer 101 to cause a series of operational steps to be performed by processor set 106 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 108 and the other storage media discussed below. The program instructions, and associated data, are accessed by processor set 106 to control and direct performance of the inventive methods. In computing environment 100, at least some of the instructions for performing the inventive methods may be stored in block 125 in persistent storage 111.

Communication fabric 109 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 110 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 110 is located in a single package and is internal to computer 101, but, alternatively or additionally, the volatile memory may be distributed over multiple packages and/or located externally with respect to computer 101.

Persistent Storage 111 is any form of non-volatile storage for computers that is now known or to be developed in the future. The non-volatility of this storage means that the stored data is maintained regardless of whether power is being supplied to computer 101 and/or directly to persistent storage 111. Persistent storage 111 may be a read only memory (ROM), but typically at least a portion of the persistent storage allows writing of data, deletion of data and re-writing of data. Some familiar forms of persistent storage include magnetic disks and solid state storage devices. Operating system 112 may take several forms, such as various known proprietary operating systems or open source Portable Operating System Interface type operating systems that employ a kernel. The code included in block 125 typically includes at least some of the computer code involved in performing the inventive methods.

Peripheral device set 113 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 114 may include components such as a display screen, speaker, microphone, wearable devices (such as goggles and smart watches), keyboard, mouse, printer, touchpad, game controllers, and haptic devices. Storage 115 is external storage, such as an external hard drive, or insertable storage, such as an SD card. Storage 115 may be persistent and/or volatile. In some embodiments, storage 115 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 116 is made up of sensors that can be used in Internet of Things applications. For example, one sensor may be a thermometer and another sensor may be a motion detector.

Network module 117 is the collection of computer software, hardware, and firmware that allows computer 101 to communicate with other computers through WAN 124. Network module 117 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 117 are performed on the same physical hardware device. In other embodiments (for example, embodiments that utilize software-defined networking (SDN)), the control functions and the forwarding functions of network module 117 are performed on physically separate devices, such that the control functions manage several different network hardware devices. Computer readable program instructions for performing the inventive methods can typically be downloaded to computer 101 from an external computer or external storage device through a network adapter card or network interface included in network module 117.

WAN 124 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 102 is any computer system that is used and controlled by an end user (for example, a customer of an enterprise that operates computer 101), and may take any of the forms discussed above in connection with computer 101. EUD 102 typically receives helpful and useful data from the operations of computer 101. For example, in a hypothetical case where computer 101 is designed to provide a recommendation to an end user, this recommendation would typically be communicated from network module 117 of computer 101 through WAN 124 to EUD 102. In this way, EUD 102 can display, or otherwise present, the recommendation to an end user. In some embodiments, EUD 102 may be a client device, such as thin client, heavy client, mainframe computer, desktop computer and so on.

Remote server 103 is any computer system that serves at least some data and/or functionality to computer 101. Remote server 103 may be controlled and used by the same entity that operates computer 101. Remote server 103 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 118 of remote server 103.

Public cloud 104 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 104 is performed by the computer hardware and/or software of cloud orchestration module 120. The computing resources provided by public cloud 104 are typically implemented by virtual computing environments that run on various computers making up the computers of host physical machine set 121, which is the universe of physical computers in and/or available to public cloud 104. The virtual computing environments (VCEs) typically take the form of virtual machines from virtual machine set 122 and/or containers from container set 123. It is understood that these VCEs may be stored as images and may be transferred among and between the various physical machine hosts, either as images or after instantiation of the VCE. Cloud orchestration module 120 manages the transfer and storage of images, deploys new instantiations of VCEs and manages active instantiations of VCE deployments. Gateway 119 is the collection of computer software, hardware, and firmware that allows public cloud 104 to communicate through WAN 124.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 105 is similar to public cloud 104, except that the computing resources are only available for use by a single enterprise. While private cloud 105 is depicted as being in communication with WAN 124 in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 104 and private cloud 105 are both part of a larger hybrid cloud.

Block 125 further includes the software components which are used to monitor and improve mental health outcomes via conversational artificial intelligence. In one embodiment, such components may be implemented in hardware. The functions discussed above performed by such components are not generic computer functions. As a result, computer 101 is a particular machine that is the result of implementing specific, non-generic computer functions.

In one embodiment, the functionality of such software components of computer 101, including the functionality for monitoring and improving mental health outcomes via conversational artificial intelligence, may be embodied in an application specific integrated circuit.

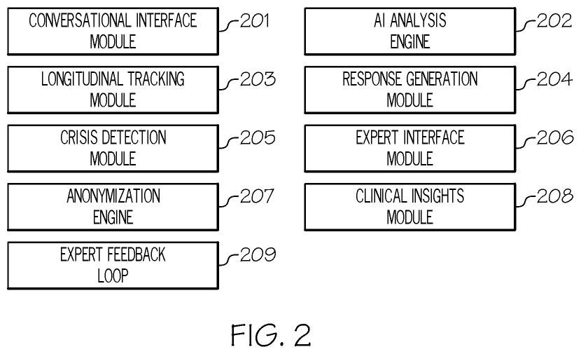

A further discussion regarding the functionality of the components used by computer 101 to monitor and improve mental health outcomes via conversational artificial intelligence is provided below in connection with FIG. 2.

FIG. 2 is a diagram of the software components used by computer 101 to monitor and improve mental health outcomes via conversational artificial intelligence in accordance with an embodiment of the present disclosure.

Referring to FIG. 2, in conjunction with FIG. 1, computer 101 includes conversational interface module 201. In one embodiment, conversational interface module 201 is configured to facilitate a natural language dialogue between a user and computer 101 by receiving conversational inputs from the user during a current session and providing personalized, empathetic responses generated by the system. A conversational input, as used herein, refers to a natural language utterance, such as a text-based message or a transcribed voice recording, received from a user via a conversational interface module that serves as the raw data source for the identification of a sentiment or a mental health indicator by AI analysis engine 202. In one embodiment, conversational interface module 201 is implemented through various platforms, including mobile applications and web-based interfaces, to ensure 24/7 accessibility for the user. In one embodiment, in addition to text-based interaction, conversational interface module 201 is configured to support voice-to-text inputs and include science-backed coping strategies or self-care suggestions as part of the transmitted response to bridge the gap between professional therapy sessions.

Computer 101 further includes AI analysis engine 202 configured to process the conversational input received by conversational interface module 201. In one embodiment, AI analysis engine 202 utilizes a large language model (LLM), such as a fine-tuned Gemma-2B-IT instruction-based model, to perform natural language understanding (NLU) on the user's input. Specifically, AI analysis engine 202 is configured to identify a sentiment and/or a mental health indicator, such as levels of stress, anxiety, or depression, by analyzing the linguistic patterns and emotional valence of the conversational input. A sentiment, as used herein, is a quantified polarity score, such as positive, negative, or neutral, determined by AI analysis engine 202 through the evaluation of the emotional valence and linguistic patterns of a user's conversational input. A mental health indicator, as used herein, is a specific clinical metric or behavioral marker, such as a measured level of stress, anxiety, or depression, identified within a conversational input to characterize a user's current psychological state. In one embodiment, AI analysis engine 202 utilizes prompt engineering to specialize responses based on a specific mental health focus, ensuring the identified indicators are contextually relevant to the user's ongoing mental health journey.

Computer 101 additionally includes longitudinal tracking module 203 configured to manage and analyze user data across a plurality of conversational sessions to monitor a user's mental health journey over time. In one embodiment, longitudinal tracking module 203 is configured to retrieve historical data points from a database, such as remote database 118, and aggregate these points with real-time indicators identified by AI analysis engine 202. In one embodiment, longitudinal tracking module 203 is further configured to generate graphical representations of this data, including, but not limited to, calendar heatmaps of symptom intensity and violin plots of mental health metrics, such as stress, depression, and anxiety levels over a period of weeks. By maintaining this continuous record, longitudinal tracking module 203 enables the system to identify cross-session patterns that assist in evaluating the longitudinal efficacy of the conversational interventions.

Furthermore, computer 101 includes response generation module 204 configured to synthesize and transmit contextually relevant and empathetic replies to the user. In one embodiment, response generation module 204 utilizes the identified mental health indicators from AI analysis engine 202 and the longitudinal historical data retrieved by longitudinal tracking module 203 to produce a personalized response. In one embodiment, response generation module 204 is configured to incorporate science-backed coping strategies and self-care suggestions into the dialogue to provide immediate support to the user. Additionally, response generation module 204 may utilize specific instruction-oriented data points to ensure that the output is non-dismissive and aligned with therapeutic best practices.

Additionally, computer 101 includes crisis detection module 205 configured to monitor conversational inputs in real-time for high-risk indicators. A high-risk indicator, as used herein, refers to a specific sentiment or mental health metric identified within a user's conversational input that signifies an acute crisis or emergency requiring the immediate execution of a crisis intervention protocol. A crisis intervention protocol, as used herein, is a programmed sequence of safety actions, such as providing emergency contact resources or sending direct notifications to a human-in-the-loop professional, that is automatically executed by the system upon the detection of a high-risk mental health indicator within a user's conversational input.

In one embodiment, crisis detection module 205 is configured to analyze the sentiment and mental health metrics identified by AI analysis engine 202 to determine if a user's input signifies an acute crisis or emergency. Upon the detection of such a high-risk indicator, crisis detection module 205 is further configured to automatically execute a crisis intervention protocol, which may include providing the user with emergency contact resources or transmitting a direct notification to a human-in-the-loop professional. This autonomous safety layer ensures that the system can respond immediately to urgent mental health needs by overriding standard conversational logic with science-backed safety procedures

Furthermore, computer 101 includes expert interface module 206 configured to facilitate communication and data exchange between the system and a professional therapist. In one embodiment, expert interface module 206 is configured to provide the professional therapist with access to user-specific clinical data, including the longitudinal historical data and graphical representations generated by longitudinal tracking module 203. In one embodiment, expert interface module 206 is configured to display data-driven insights and cross-session patterns, such as symptom intensity trends, to augment the therapist's clinical workflow. Additionally, expert interface module 206 may include a secure portal through which the professional therapist can provide evaluative feedback on AI-generated responses, thereby enabling the “human-in-the-loop” oversight required to align the system with professional therapeutic standards.

Computer 101 further includes anonymization engine 207 configured to safeguard user privacy and ensure the ethical handling of sensitive mental health data. In one embodiment, anonymization engine 207 is configured to process the conversational input and the identified mental health indicators to remove or obscure personally identifiable information (PII) prior to storing said data in a database, such as remote database 118. By scrubbing unique identifiers while preserving the clinical utility of the linguistic and emotional metrics, anonymization engine 207 enables the secure generation of longitudinal insights and the sharing of data with professional therapists via expert interface module 206. This automated privacy layer ensures that the system remains compliant with data protection standards while maintaining the integrity of the user's longitudinal mental health record.

Computer 101 additionally includes clinical insights module 208 configured to transform complex longitudinal data into actionable intelligence for a healthcare provider. In one embodiment, clinical insights module 208 is configured to analyze the updated longitudinal historical data stored in remote database 118 to identify at least one cross-session pattern or trend in the user's mental health metrics. Clinical insights module 208 may be further configured to generate data-driven insights based on these identified patterns, providing a professional therapist with a high-level overview of a user's progress, such as improvements in emotional regulation or the frequency of specific symptom triggers. By synthesizing raw conversational data into clinical observations, clinical insights module 208 augments the therapist's ability to tailor treatment plans and monitor the long-term efficacy of the digital interventions.

Furthermore, computer 101 includes expert feedback loop 209 configured to facilitate the iterative refinement of the large language model (LLM) based on professional clinical oversight. In one embodiment, expert feedback loop 209 is configured to receive evaluations of personalized responses from a professional therapist via expert interface module 206. In one embodiment, expert feedback loop 209 is further configured to utilize these evaluations, which may include ratings, corrections, or clinical alignment notes, to further tune the LLM. This “human-in-the-loop” mechanism ensures that the conversational agent's future responses are increasingly aligned with clinical standards and therapeutic best practices, thereby enhancing the overall efficacy and safety of the system

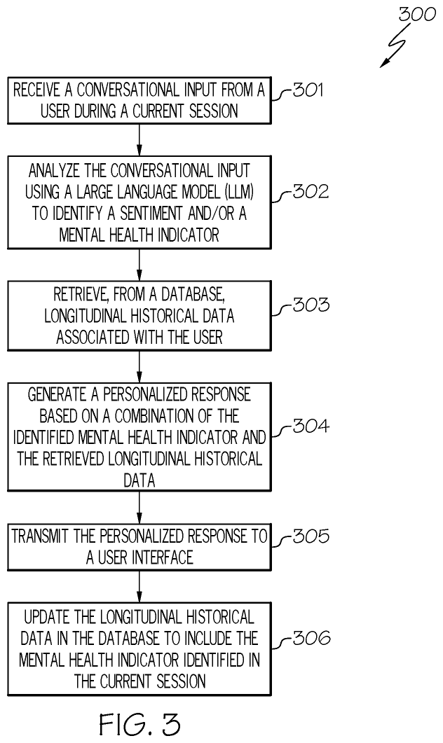

A discussion regarding the method for monitoring and improving mental health outcomes via conversational artificial intelligence using the software components discussed above is provided below in connection with FIG. 3.

FIG. 3 is a flowchart of a method 300 for monitoring and improving mental health outcomes via conversational artificial intelligence in accordance with an embodiment of the present disclosure.

Referring now to FIG. 3, in conjunction with FIGS. 1-2, in step 301, conversational interface module 201 receives a conversational input from a user during a current session. As stated above, a conversational input, as used herein, refers to a natural language utterance, such as a text-based message or a transcribed voice recording, received from a user via a conversational interface module that serves as the raw data source for the identification of a sentiment or a mental health indicator by AI analysis engine 202.

In one embodiment, the conversational input includes natural language text entered by the user via a user interface of end user device 102. In another embodiment, the conversational input may include an audio stream that is converted to text using a speech-to-text processor within conversational interface module 201. The conversational input may be received in real-time as part of an active therapeutic dialogue or as a standalone entry. Upon receipt, conversational interface module 201 may perform initial pre-processing, such as tokenization or language identification, to prepare the input for analysis by the downstream software components of computer 101.

In one embodiment, the receipt of the conversational input in step 301 includes establishing a secure communication session between end user device 102 and computer 101 using standard encryption protocols (e.g., SSL/TLS). The conversational input may include metadata associated with the user session, such as a timestamp, a unique session identifier, and device telemetry data, which is temporarily buffered in memory (e.g., memory 110) for synchronized processing. Furthermore, the pre-processing performed by conversational interface module 201 may include the application of a filter or a preliminary intent-recognition algorithm to categorize the input (e.g., as a greeting, a factual query, or a subjective emotional statement) before it is passed to AI analysis engine 202 for deeper sentiment and indicator extraction. This initial categorization allows the system to prioritize the computational resources of processor set 106 based on the complexity or urgency of the received natural language string.

In step 302, AI analysis engine 202 analyzes the conversational input using a large language model (LLM) to identify a sentiment and/or a mental health indicator. A sentiment, as used herein, is a quantified polarity score, such as positive, negative, or neutral, determined by AI analysis engine 202 through the evaluation of the emotional valence and linguistic patterns of a user's conversational input. A mental health indicator, as used herein, is a specific clinical metric or behavioral marker, such as a measured level of stress, anxiety, or depression, identified within a conversational input to characterize a user's current psychological state.

In one embodiment, the LLM is a fine-tuned, instruction-based model, such as the Gemma-2B-IT model, which has been optimized for natural language understanding (NLU) within a clinical context. In one embodiment, the LLM has been trained on a dataset that includes therapist-validated patient-question and response pairs. AI analysis engine 202 processes the pre-processed natural language tokens from step 301 by passing them through a series of neural network layers (e.g., transformer blocks) to evaluate the linguistic structure, context, and emotional valence of the user's input.

In one embodiment, AI analysis engine 202 analyzes the conversational input using a large language model (LLM) to identify a sentiment and/or a mental health indicator by applying a specific instruction-based prompt to the LLM trained on the therapist-validated dataset to guide the extraction of mental health metrics. For example, AI analysis engine 202 may utilize prompt engineering to task the model with identifying specific indicators, such as stress, anxiety, or depression levels, on a predefined numerical scale (e.g., 1 to 10). In one embodiment, AI analysis engine 202 identifies the “sentiment” by calculating a polarity score (e.g., positive, negative, or neutral) and identifies “mental health indicators” by detecting keywords, semantic clusters, or behavioral markers associated with clinical distress.

Furthermore, in one embodiment, AI analysis engine 202 analyzes the conversational input using a large language model (LLM) to identify a sentiment and/or a mental health indicator using specialized attention mechanisms within the LLM to weigh the significance of certain phrases relative to the user's stated history, such as by increasing the attention weights for tokens associated with previously identified symptom triggers. By identifying these indicators in real-time, AI analysis engine 202 produces a structured data output, such as a JSON object, containing the quantified sentiment and indicator values. This structured output is then temporarily stored in a memory (e.g., memory 110) to serve as the basis for both the immediate response generation and the long-term longitudinal tracking performed in subsequent steps.

In step 303, longitudinal tracking module 203 retrieves from a database (e.g., database 118) longitudinal historical data associated with the user. Longitudinal historical data, as used herein, refers to a time-series collection of previously identified sentiments and mental health indicators, along with associated session metadata, captured across a plurality of prior conversational sessions between the user and computer 101. In one embodiment, this data is stored as indexed records within a relational or non-relational database structure, where each record is mapped to a unique user identifier to ensure continuity of the clinical profile.

In one embodiment, longitudinal tracking module 203 retrieves from a database (e.g., database 118) longitudinal historical data associated with the user by executing a query (e.g., a SQL or NoSQL query) via a secure network interface to extract a subset of historical data within a defined temporal window, such as the previous thirty days. In one embodiment, longitudinal tracking module 203 is configured to pull raw numerical values representing symptom intensity as well as qualitative linguistic markers that were previously flagged by AI analysis engine 202. This retrieval allows the system to establish a “baseline” psychological state for the user, which is necessary for identifying deviations or improvements in the current session.

Furthermore, in one embodiment, longitudinal tracking module 203 decrypts the historical data using a session-specific key, ensuring that the sensitive mental health record is only readable within the secure execution environment of processor set 106. Once retrieved and decrypted, the longitudinal historical data is loaded into a structured data format (e.g., a data frame or array) within memory 110. This enables longitudinal tracking module 203 to perform comparative analysis between the historical baseline and the real-time indicators identified in step 302, facilitating the generation of the graphical representations, such as calendar heatmaps or violin plots, described in the following steps.

In step 304, response generation module 204 generates a personalized response based on a combination of the identified mental health indicator and the retrieved longitudinal historical data. A personalized response, as used herein, refers to a contextually-aware natural language output that is dynamically adjusted based on a user's current emotional state and their established psychological baseline to provide empathetic support and clinical relevance. In one embodiment, the personalized response is synthesized by passing the structured data output from step 302 and the historical data frames from step 303 into a response-generation prompt template within the LLM.

In one embodiment, response generation module 204 generates a personalized response by calculating a “delta” or variance between the current mental health indicator (e.g., a current anxiety score of 8) and the historical average (e.g., a 30-day mean anxiety score of 4). Based on this variance, response generation module 204 is configured to select from a library of science-backed coping strategies, such as grounding exercises or cognitive reframing techniques, that are specifically mapped to high-variance states. This ensures that the response is not merely a generic greeting but a targeted intervention that acknowledges the user's specific longitudinal trend.

Furthermore, in one embodiment, there is an “empathy-tuning” phase where response generation module 204 applies instruction-oriented data points to the LLM to modulate the tone and emotional valence of the output. For example, if the current sentiment is identified as highly negative, response generation module 204 triggers a “non-dismissive” constraint that prevents the generation of toxic positivity and instead prioritizes validating language. The finalized personalized response is then formatted for delivery to conversational interface module 201, which transmits the response to end user device 102 via a secure communication channel, thereby completing the real-time feedback loop.

In step 305, response generation module 204 transmits the personalized response to a user interface of end user device 102. In one embodiment, the transmission involves encapsulating the natural language output, along with any associated graphical representations (e.g., the calendar heatmaps or violin plots generated by longitudinal tracking module 203), into a secure data packet for delivery via a network interface. In one embodiment, conversational interface module 201 then renders this data within a mobile application or web-based portal, presenting the personalized response in an empathetic, conversational format designed to bridge the gap between professional therapy sessions.

In one embodiment, the transmission includes a latency-management protocol to ensure that the AI-generated response is delivered in a timeframe consistent with human-like conversation. In one embodiment, response generation module 204 is configured to stream the natural language output in real-time, allowing the user to begin reading the response as it is being synthesized by the LLM. Furthermore, in one embodiment, response generation module 204 includes the concurrent transmission of metadata that triggers specific UI elements on end user device 102, such as highlighting science-backed coping strategies or providing clickable links to crisis intervention resources if a high-risk indicator was previously flagged.

Additionally, in one embodiment, response generation module 204 transmits the personalized response to a user interface of end user device 102 by utilizing a confirmation handshake between end user device 102 and computer 101 to verify the successful receipt and display of the personalized response. Upon successful delivery, the system is configured to transition from the active dialogue phase to a data-retention phase, where the current session metrics are prepared for anonymization and storage. This ensures that the user's immediate emotional needs are met before the system performs the background computational tasks required to maintain the longitudinal mental health record.

In step 306, longitudinal tracking module 203, supported by anonymization engine 207, updates the longitudinal historical data in the database (e.g. database 118) to include the mental health indicator identified in the current session.

In one embodiment, prior to the update, anonymization engine 207 processes the conversational input and the identified mental health indicators to remove or obscure personally identifiable information (PII). This scrubbing process ensures that the clinical utility of the session, such as the quantified stress or anxiety metrics, is preserved for longitudinal tracking while the user's privacy is maintained within the permanent record stored in remote database 118.

In one embodiment, longitudinal tracking module 203 updates the longitudinal historical data in the database (e.g. database 118) to include the mental health indicator identified in the current session by executing a write operation (e.g., a SQL “INSERT” or “UPDATE” command) to append the anonymized session metrics to the user's unique time-series profile. This update includes the real-time indicators identified by AI analysis engine 202, the associated timestamps, and any metadata regarding the efficacy of the personalized response provided in step 305. By updating the historical baseline in this manner, longitudinal tracking module 203 ensures that subsequent conversational sessions (e.g., in a future iteration of step 301) have access to the most recent clinical context.

Furthermore, the updating of the longitudinal historical data in the database (e.g. database 118) involves the automatic generation of updated graphical representations by longitudinal tracking module 203. For example, the system may update a stored calendar heatmap or violin plot to reflect the intensity and valence of the current session's metrics. These updated visualizations and the underlying anonymized data are then made available to a professional therapist via expert interface module 206, thereby completing the “human-in-the-loop” feedback cycle and enabling the long-term monitoring of the user's mental health journey.

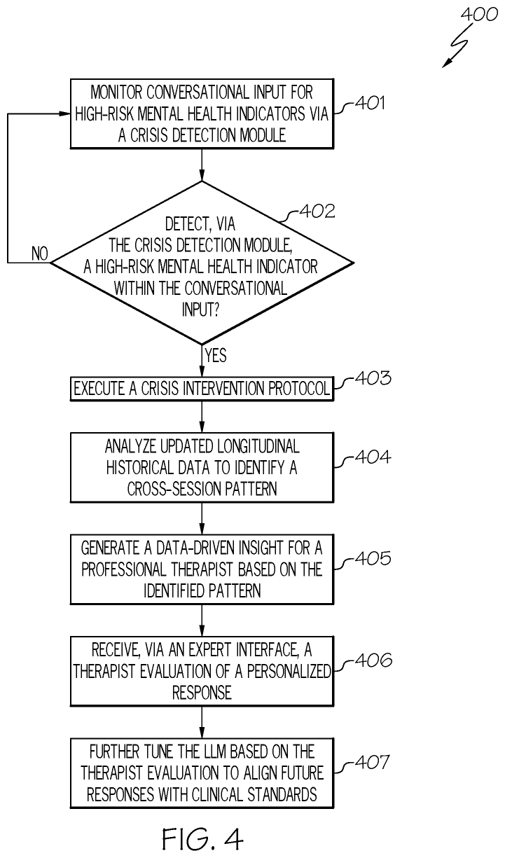

While FIG. 3 illustrates the operational flow for monitoring and improving mental health outcomes, the technical efficacy of the system is further enhanced by the generation and display of specific longitudinal data visualizations. These visualizations allow both the user and a professional therapist to identify non-obvious patterns in psychological states that may not be apparent during a single conversational session. A discussion regarding the specific graphical representations of the longitudinal historical data, such as calendar heatmaps and symptom distribution plots, is provided below in connection with FIG. 4.

FIG. 4 is a flowchart of a method 400 for implementing an exemplary user interface (UI) view of a longitudinal mental health dashboard in accordance with an embodiment of the present disclosure.

Referring to FIG. 4, in conjunction with FIGS. 1-3, in step 401, crisis detection module 205 monitors the conversational input for high-risk, mental health indicators. As discussed above, a high-risk mental health indicator, as used herein, refers to a specific sentiment or mental health metric identified within a user's conversational input that signifies an acute crisis or emergency requiring the immediate execution of a crisis intervention protocol

In one embodiment, the monitoring of the conversational input in step 401 occurs in parallel with the AI analysis performed in step 302. In one embodiment, crisis detection module 205 is configured to scan the structured data output and natural language tokens for predefined trigger patterns, such as semantic clusters associated with self-harm, ideation, or acute clinical distress. Unlike the general sentiment analysis, the identification of a high-risk mental health indicator in step 401 utilizes a specialized classification layer within the LLM or a secondary rules-based engine designed for high-precision detection of emergency markers.

Furthermore, crisis detection module 205 monitors the conversational input for high-ris, mental health indicators by comparing the identified indicator against a severity threshold stored in memory 110. If the intensity of a detected indicator exceeds this threshold (e.g., a score of 9 or 10 on a 10-point distress scale), then crisis detection module 205 is enabled to trigger a high-priority interrupt signal to processor set 106. This interrupt signal prioritizes the execution of the crisis intervention protocol over the standard response generation logic of step 304, ensuring that life-safety resources are deployed without the latency associated with standard empathetic dialogue synthesis.

In one embodiment, crisis detection module 205 utilizes a “false-positive” filter to distinguish between figurative language and genuine acute crisis. By utilizing the longitudinal historical data retrieved in step 303, crisis detection module 205 can contextualize the current high-risk indicator against the user's baseline linguistic patterns. If an acute crisis is confirmed, the module flags the session record with a high-risk metadata tag, which is subsequently used by expert interface module 206 to alert a professional therapist in real-time, thereby enabling immediate human intervention.

In step 402, crisis detection module 205 determines whether it detected a high-risk mental health indicator within the conversational input. This determination functions as a real-time gate within the processing pipeline of computer 101.

In one embodiment, crisis detection module 205 evaluates the metadata tag or “flag” generated during the monitoring of step 401. If the flag indicates the absence of a high-risk mental health indicator (i.e., crisis detection module 205 does not detect a high-risk mental health indicator within the conversational input), then crisis detection module 205 continues to monitor the conversational input for high-risk mental health indicators in step 401 to maintain standard conversational monitoring and response generation.

If, however, crisis detection module 205 detects a high-risk mental health indicator within the conversational input, then, in step 403, crisis detection module 205 executes a crisis intervention protocol. As stated above, a crisis intervention protocol, as used herein, refers to a programmed sequence of safety actions, such as providing emergency contact resources or sending direct notifications to a human-in-the-loop professional, that is automatically executed by the system upon the detection of a high-risk mental health indicator within a user's conversational input.

In one embodiment, crisis detection module 205 executes the crisis intervention protocol by implementing the immediate suspension of the standard LLM-based response synthesis to minimize computational latency. Crisis detection module 205 retrieves a set of pre-validated emergency response templates from a non-volatile memory (e.g., database 118) and loads them into volatile memory 110 for immediate transmission. These templates may include localized emergency phone numbers, links to immediate counseling services, or grounding exercises specifically indexed for high-acute distress.

Furthermore, crisis detection module 205 executes the crisis intervention protocol by utilizing a multi-channel notification sequence. In such an embodiment, crisis detection module 205 generates an encrypted alert packet containing the user's recent anonymized indicators and transmits this packet to expert interface module 206. This triggers a “high-priority” visual or auditory alert on a professional therapist's workstation, enabling a human-in-the-loop to review the conversational context in real-time. By automating this escalation, the crisis intervention protocol ensures that the technical system bridges the gap between automated support and professional clinical intervention during critical windows of user need.

In step 404, clinical insights module 208 analyzes the updated longitudinal historical data to identify a cross-session pattern. A cross-session pattern, as used herein, refers to a statistically significant trend or correlation identified across a plurality of conversational sessions, such as a multi-day increase in anxiety markers, a cyclical pattern of depressive symptoms, or a positive correlation between specific coping strategies and improved sentiment scores. In one embodiment, clinical insights module 208 identifies these patterns by applying time-series analysis algorithms (e.g., moving averages or seasonal decomposition) to the anonymized metrics stored in database 118.

In one embodiment, clinical insights module 208 analyzes the updated longitudinal historical data to identify a cross-session pattern by calculating a “velocity of change” for specific mental health indicators. In one embodiment, clinical insights module 208 compares the slope of the user's current emotional trajectory against a historical baseline to determine if a user's psychological state is stabilizing, deteriorating, or improving. This analysis allows the system to distinguish between a single “bad day” and a persistent downward trend that may require a change in the therapeutic approach. In one embodiment, the identified patterns are stored as high-level metadata within volatile memory 110 for immediate use in the visualization and reporting steps.

Furthermore, clinical insights module 208 identifies the “trigger-response” pairs. A “trigger-response” pair, as used herein, refers to a statistically significant correlation identified by clinical insights module 208 between a specific conversational topic or environmental factor (the trigger) and a subsequent, measurable shift in a user's sentiment or mental health indicators (the response) across a plurality of sessions. For example, clinical insights module 208 may correlate the mention of specific topics (e.g., work-related stress) with subsequent drops in sentiment polarity across multiple weeks. By identifying these latent patterns, clinical insights module 208 provides the “intelligence” layer that powers the specialized graphical representations, such as the calendar heatmaps or violin plots, by providing the raw data points necessary to populate those complex visual distributions.

In step 405, clinical insights module 208 generates a data-driven insight for a professional therapist based on the identified pattern. A data-driven insight, as used herein, refers to a synthesized clinical observation or actionable recommendation derived from the statistical analysis of longitudinal historical data, which highlights specific deviations from a user's psychological baseline or correlates triggers with symptom intensity. In one embodiment, the data-driven insight is formatted as a natural language summary or a prioritized alert that is optimized for review within expert interface module 206.

In one embodiment, clinical insights module 208 generates the data-driven insight by passing the cross-session patterns and “trigger-response” pairs identified in step 404 into a functional component of clinical insights module 208. This functional component compares the current longitudinal trends against a library of clinical markers stored in database 118. For example, if clinical insights module 208 identifies a “velocity of change” that indicates a rapid decrease in sentiment polarity over a seven-day period, then the data-driven insight may include a “stability warning” and a suggestion to prioritize the user's next scheduled therapy session.

Furthermore, clinical insights module 208 generates a data-driven insight by generating comparative insights that contrast a user's self-reported feelings with the objective linguistic markers identified by AI analysis engine 202. If a significant discrepancy is detected (e.g., the user reports “feeling fine” but the linguistic markers show high cognitive distortion), clinical insights module 208 generates an “insight flag” to alert the therapist to a potential lack of user insight or masking of symptoms. By synthesizing these complex data points into a concise insight, the system enables the professional therapist to make more informed clinical decisions without manually auditing the raw conversational logs from every session.

In step 406, expert feedback loop 209 receives, via an expert interface, a therapist evaluation of a personalized response. The expert interface, as used herein, refers to a secure, web-based or application-based graphical user interface (GUI) provided by expert interface module 206 that allows a professional therapist to review conversational logs, identified mental health indicators, and the system-generated personalized responses. The therapist evaluation of a personalized response, as used herein, refers to a qualitative or quantitative assessment provided by the professional therapist regarding the clinical accuracy, empathetic tone, or therapeutic appropriateness of the response generated in step 304.

In one embodiment, the expert interface provided by expert interface module 206 includes a set of interactive UI elements, such as Likert scales, binary “thumbs-up/thumbs-down” toggles, or text entry fields for specific clinical corrections, which are captured by expert feedback loop 209. Furthermore, in one embodiment, expert feedback loop 209 is configured to capture these inputs and map them to the specific session ID and metadata associated with the personalized response. This allows the system to associate the therapist's expert judgment directly with the linguistic markers and historical context that triggered that specific response, creating a high-fidelity training pair for future system optimization.

Furthermore, in one embodiment, the therapist evaluation is transmitted from expert interface module 206 back to AI analysis engine 202 or a centralized model-tuning repository in database 118. In one embodiment, this evaluation acts as a “ground truth” signal that is used to refine the prompt templates or fine-tune the underlying LLM parameters. By integrating this professional feedback, expert feedback loop 209 enables the system to iteratively improve its empathetic delivery and clinical alignment, ensuring that the machine-generated support remains consistent with professional standards of care.

In step 407, expert feedback loop 209 further tunes the LLM based on the therapist evaluation to align future responses with clinical standards.

In one embodiment, this tuning process includes a closed-loop optimization cycle where the qualitative and quantitative assessments received in step 406 are converted into a structured reward signal. This reward signal is then utilized by a fine-tuning module within expert feedback loop 209 to adjust the weights or parameters of the underlying LLM (e.g., via a Low-Rank Adaptation (LoRA) or similar parameter-efficient fine-tuning technique) to favor response patterns that the professional therapist has rated as clinically appropriate.

In one embodiment, the tuning in step 407 involves the iterative refinement of the system's “system prompts” or “persona instructions.” In one embodiment, expert feedback loop 209 analyzes therapist evaluations to identify recurring clinical corrections (e.g., a need for more “validation” or less “problem-solving” in certain contexts). Based on this analysis, the system automatically updates the hidden instructions provided to response generation module 204. This ensures that in subsequent conversational sessions (e.g., a future iteration of step 301), the natural language synthesis is pre-conditioned to adhere to the corrected therapeutic style without requiring manual re-coding.

Furthermore, expert feedback loop 209 further tunes the LLM based on the therapist evaluation to align future responses with clinical standards by including the population of a “Gold Standard” dataset within database 118. This Gold Standard dataset consists of conversational inputs paired with therapist-approved “ideal” responses. During the tuning process, expert feedback loop 209 uses this dataset to perform periodic validation checks, ensuring that the system's performance does not “drift” away from established clinical standards over time. By implementing this continuous alignment, expert feedback loop 209 creates a self-optimizing technical environment that bridges the gap between automated AI scale and professional clinical expertise.

Furthermore, the principles of the present disclosure improve the technology or technical field involving automated mental health monitoring.

As discussed above, digital mental health solutions, and specifically conversational artificial intelligence (AI) agents, have emerged as a promising means to address significant barriers in mental health care. Traditional therapy often faces challenges, such as high costs, long wait times for appointments, limited availability of practitioners, and persistent social stigma that may prevent individuals from seeking help. Conversational AI, typically implemented as chatbots, has the potential to bridge the gap between mental health needs and available resources by providing 24/7 accessible, affordable, and scalable assistance. Despite these advantages, existing mental health AI models often suffer from critical limitations that hinder their therapeutic effectiveness. Many current systems utilize a “one-size-fits-all” approach that lacks personalized engagement and fails to track a user's progress over time. Furthermore, typical conversational agents often lack genuine empathy and emotional nuance, which can lead to slow or unnatural responses that alienate users, especially during acute crises or complex mental health emergencies. Moreover, existing technologies often operate as isolated tools rather than integrated components of a broader clinical workflow. There is frequently a lack of “human-in-the-loop” oversight, meaning that valuable insights from chat histories are not effectively shared with professional therapists to augment their treatment plans. Concerns regarding data privacy, potential algorithmic bias, and a lack of longitudinal studies further complicate the trustworthiness and safety of these digital interventions. Consequently, there is a need for a comprehensive conversational AI framework that provides human-like, personalized support while maintaining a high standard of clinical alignment and ethical safety. Such a system would ideally integrate expert therapist feedback, offer science-backed coping strategies, and utilize data-driven insights to help both users and practitioners track mental health journeys over time without replacing the essential role of the human therapist.

Embodiments of the present disclosure improve such technology by receiving a conversational input from a user via a conversational interface module. The conversational input is then analyzed using an AI analysis engine to identify a sentiment and/or a mental health indicator. An AI analysis engine, as used herein, refers to a large language model (LLM) or specialized neural network configured to perform natural language understanding (NLU) to identify psychological markers within text. A sentiment, as used herein, is a quantified polarity score, such as positive, negative, or neutral, determined by the AI analysis engine through the evaluation of the emotional valence and linguistic patterns of a user's conversational input. A mental health indicator, as used herein, is a specific clinical metric or behavioral marker, such as a measured level of stress, anxiety, or depression, identified within a conversational input to characterize a user's current psychological state. Longitudinal historical data associated with the user is then retrieved from a database, where the longitudinal historical data includes data points from the previous conversational sessions. Longitudinal historical data, as used herein, refers to a persistent, anonymized record of quantified psychological metrics tracked over a plurality of conversational sessions to establish a clinical baseline. A personalized response is then generated based on the identified mental health indicator and the retrieved longitudinal historical data. A personalized response, as used herein, refers to a contextually-aware natural language output that is dynamically adjusted based on a user's current emotional state and their established psychological baseline to provide empathetic support and clinical relevance. In one embodiment, the personalized response is synthesized by passing the identified mental health indicator and the longitudinal historical data into a response-generation prompt template within the LLM. The personalized response is then transmitted to a user interface. The longitudinal historical data in the database is then updated to include the mental health indicator identified in the current session. Furthermore, an expert interface is utilized to receive a therapist evaluation of the personalized response to further tune the AI analysis engine via an expert feedback loop. In this manner, the system creates a self-optimizing technical environment where the automated generation of responses is continuously refined by professional expertise. Furthermore, in this manner, there is an improvement in the technical field involving automated mental health monitoring.

The technical solution provided by the present disclosure cannot be performed in the human mind or by a human using a pen and paper. That is, the technical solution provided by the present disclosure could not be accomplished in the human mind or by a human using a pen and paper in any reasonable amount of time and with any reasonable expectation of accuracy without the use of a computer.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A computer-implemented method for providing augmented mental health support, the method comprising:
receiving a conversational input from a user during a current session;
analyzing said conversational input using a large language model (LLM) to identify a mental health indicator;
retrieving, from a database, longitudinal historical data associated with said user, wherein said longitudinal historical data comprises data points from a plurality of previous conversational sessions;
generating a personalized response based on a combination of said identified mental health indicator and said retrieved longitudinal historical data;
transmitting said personalized response to a user interface; and
updating said longitudinal historical data in said database to include said mental health indicator identified in said current session.

2. The method as recited in claim 1 further comprising:
analyzing said updated longitudinal historical data to identify a cross-session pattern; and
generating a data-driven insight for a professional therapist based on said identified cross-session pattern.

3. The method as recited in claim 1 further comprising:
generating a graphical representation of said longitudinal historical data, wherein said graphical representation comprises at least one of a calendar heatmap of symptom intensity and a violin plot of longitudinal mental health metrics.

4. The method as recited in claim 1, wherein said LLM is an instruction-based model trained on a dataset comprising a plurality of therapist-validated patient-question and response pairs.

5. The method as recited in claim 1 further comprising:
detecting, via a crisis detection module, a high-risk mental health indicator within said conversational input; and
executing a crisis intervention protocol in response to said detection, wherein said crisis intervention protocol comprises providing at least one of an emergency contact resource and a direct notification to a human-in-the-loop professional.

6. The method as recited in claim 1 further comprising:
receiving, via an expert interface, an evaluation of said personalized response from a professional therapist; and
further tuning said LLM based on said evaluation to align future responses with clinical standards.

7. The method as recited in claim 1 further comprising:
anonymizing said conversational input and said identified mental health indicator prior to storing said conversational input and said identified mental health indicator in said database.

8. A computer program product for providing augmented mental health support, the computer program product comprising one or more computer readable storage mediums having program code embodied therewith, the program code comprising programming instructions for:
receiving a conversational input from a user during a current session;
analyzing said conversational input using a large language model (LLM) to identify a mental health indicator;
retrieving, from a database, longitudinal historical data associated with said user, wherein said longitudinal historical data comprises data points from a plurality of previous conversational sessions;
generating a personalized response based on a combination of said identified mental health indicator and said retrieved longitudinal historical data;
transmitting said personalized response to a user interface; and
updating said longitudinal historical data in said database to include said mental health indicator identified in said current session.

9. The computer program product as recited in claim 8, wherein the program code further comprises the programming instructions for:
analyzing said updated longitudinal historical data to identify a cross-session pattern; and
generating a data-driven insight for a professional therapist based on said identified cross-session pattern.

10. The computer program product as recited in claim 8, wherein the program code further comprises the programming instructions for:
generating a graphical representation of said longitudinal historical data, wherein said graphical representation comprises at least one of a calendar heatmap of symptom intensity and a violin plot of longitudinal mental health metrics.

11. The computer program product as recited in claim 8, wherein said LLM is an instruction-based model trained on a dataset comprising a plurality of therapist-validated patient-question and response pairs.

12. The computer program product as recited in claim 8, wherein the program code further comprises the programming instructions for:
detecting, via a crisis detection module, a high-risk mental health indicator within said conversational input; and
executing a crisis intervention protocol in response to said detection, wherein said crisis intervention protocol comprises providing at least one of an emergency contact resource and a direct notification to a human-in-the-loop professional.

13. The computer program product as recited in claim 8, wherein the program code further comprises the programming instructions for:
receiving, via an expert interface, an evaluation of said personalized response from a professional therapist; and
further tuning said LLM based on said evaluation to align future responses with clinical standards.

14. The computer program product as recited in claim 8, wherein the program code further comprises the programming instructions for:
anonymizing said conversational input and said identified mental health indicator prior to storing said conversational input and said identified mental health indicator in said database.

15. A system, comprising:
a memory for storing a computer program for providing augmented mental health support; and
a processor connected to the memory, wherein the processor is configured to execute program instructions of the computer program comprising:
receiving a conversational input from a user during a current session;
analyzing said conversational input using a large language model (LLM) to identify a mental health indicator;
retrieving, from a database, longitudinal historical data associated with said user, wherein said longitudinal historical data comprises data points from a plurality of previous conversational sessions;
generating a personalized response based on a combination of said identified mental health indicator and said retrieved longitudinal historical data;
transmitting said personalized response to a user interface; and
updating said longitudinal historical data in said database to include said mental health indicator identified in said current session.

16. The system as recited in claim 15, wherein the program instructions of the computer program further comprise:
analyzing said updated longitudinal historical data to identify a cross-session pattern; and
generating a data-driven insight for a professional therapist based on said identified cross-session pattern.

17. The system as recited in claim 15, wherein the program instructions of the computer program further comprise:
generating a graphical representation of said longitudinal historical data, wherein said graphical representation comprises at least one of a calendar heatmap of symptom intensity and a violin plot of longitudinal mental health metrics.

18. The system as recited in claim 15, wherein said LLM is an instruction-based model trained on a dataset comprising a plurality of therapist-validated patient-question and response pairs.

19. The system as recited in claim 15, wherein the program instructions of the computer program further comprise:
detecting, via a crisis detection module, a high-risk mental health indicator within said conversational input; and
executing a crisis intervention protocol in response to said detection, wherein said crisis intervention protocol comprises providing at least one of an emergency contact resource and a direct notification to a human-in-the-loop professional.

20. The system as recited in claim 15, wherein the program instructions of the computer program further comprise:
receiving, via an expert interface, an evaluation of said personalized response from a professional therapist; and
further tuning said LLM based on said evaluation to align future responses with clinical standards.
